(19) United States
(12) Patent Application Publication
(10) Pub. No.: US 2021/0068733 A1
(43) Pub. Date: Mar. 11, 2021

(54) METHODS AND SYSTEMS FOR SELF-ADMINISTERED MEASUREMENT OF CRITICAL FLICKER FREQUENCY (CFF)

(71) Applicants: United States Government as Represented by the Department of Veterans Affairs, Washington, DC (US); University of Washington, Seattle, WA (US)

(72) Inventors: George IOANNOU, Seattle, WA (US); James FOGARTY, Seattle, WA (US); Jasmine ZIA, Seattle, WA (US); Rafal KOCIELNIK, Seattle, WA (US); Ravi KARKAR, Seattle, WA (US); Sean MUNSON, Seattle, WA (US); Xiaoyi ZHANG, Seattle, WA (US)

(21) Appl. No.: 17/014,938

(22) Filed: Sep. 8, 2020

Related U.S. Application Data
(60) Provisional application No. 62/897,145, filed on Sep. 6, 2019.

(51) Int. Cl. A61B 5/16; A61B 5/00

(52) U.S. Cl. CPC A61B 5/161; A61B 5/4244; A61B 5/4064; A61B 3/0008; A61B 5/6898; A61B 5/0002; A61B 5/7282

(57) ABSTRACT

Methods, systems, and apparatuses are described causing light to be emitted, causing a frequency at which the light is emitted to vary, receiving, based on the frequency variation, a user input, determining a critical flicker frequency (CFF) corresponding to the user input, and determining, based on the CFF, a disease state.

### CROSS REFERENCE TO RELATED APPLICATION

This Application claims the benefit of U.S. application Ser. No. 62/897,145 filed Sep. 6, 2019, which is hereby incorporated by reference in its entirety.

### BACKGROUND

Critical flicker frequency (CFF) is the minimum frequency at which a flickering light source appears fused to an observer. Measuring CFF can support early diagnosis of minimal hepatic encephalopathy (MHE), a condition affecting up to 80% of people with cirrhosis of the liver. However, measuring CFF currently requires specialized equipment, such as the Lafayette Flicker Fusion System (FFS, Lafayette Instrument Company, Lafayette, Ind.). To date, such specialized equipment has been used mostly as a research tool and is not available in routine clinical practice. As such, adoption of CFF measurement in clinical practice has been hampered by the cost of a device for measuring CFF and the need for specialized training to administer the test.

### SUMMARY

Methods, systems, and apparatuses are described for determining a critical flicker frequency wherein a light is caused to be emitted and wherein the frequency at which the light is emitted is caused to vary, receiving, based on the frequency variation, a user input, determining a critical flicker frequency (CFF) corresponding to the user input, and determining, based on the CFF, a disease state.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

To easily identify the discussion of any particular element or act, the most significant digit or digits in a reference number refer to the figure number in which that element is first introduced.

FIG. 1 illustrates an exemplary system;

FIG. 2 illustrates an exemplary electronic device;

FIG. 3A illustrates an exemplary lighting device;

FIG. 3B illustrates an exemplary light source recess;

FIG. 4 illustrates an exemplary system;

FIG. 5 illustrates an exemplary system;

FIG. 6 illustrates an exemplary process;

FIG. 7 illustrates an exemplary process;

FIG. 8 illustrates exemplary results;

FIG. 9A illustrates an exemplary process;

FIG. 9B illustrates exemplary measurements

FIG. 10 illustrates an exemplary method;

FIG. 11 illustrates exemplary data;

FIG. 12 illustrates exemplary data; and

FIG. 13 illustrates exemplary data.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application, including, but not limited to, steps in disclosed methods. Thus, if there are a variety of additional steps that can be performed, it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized, including hard disks, CD-ROMs, optical storage devices, or magnetic storage devices.

Embodiments of the methods and systems are described below with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

Critical flicker frequency (CFF) is the minimum frequency at which a flickering light source appears fused to an observer. Thus, CFF represents a threshold at which the light is seen half the time as flickering and half the time as fused. Measuring CFF can support early diagnosis of a disease state, such as the existence of minimal hepatic encephalopathy (MHE), a condition affecting up to 80% of people with cirrhosis of the liver. Multiple studies have established that a healthy CFF of 40-45 Hz is reduced to <39 Hz in people with MHE. CFF has been shown to accurately detect MHE and, more importantly, to independently predict overall survival. The accuracy of CFF in diagnosing MHE has been reported to be 80%, with a sensitivity and specificity of 65% and 91%.

A discrimination method in which flicker frequencies of a light are controlled and a viewer watches the flickering light at a fixed distance using a CFF threshold may rely on the degree of decrease of the CFF threshold to determine a person's level of visual perception. When an actual measurement is performed, the flicker frequency of a light source is gradually increased until the viewer feels that the light source is not flickering. This frequency is referred as the CFF threshold. In a similar respect the flicker frequency of the light source may be gradually decreased until the viewer feels that the light source is fused (e.g., not flickering). Likewise, this frequency is also referred to as the CFF threshold. The mathematical average of the flicker frequencies at the two points may be used to represent the CFF value of the measurement (e.g., the CFF measurement).

Measuring CFF may incorporate an appropriate threshold detection algorithm. The threshold detection algorithm may implement the method of limits, which focuses on the influence and relationship between stimuli and the sensation and perception of these stimuli by an individual. For example, a stimulus (e.g., light in the case of CFF) is presented, and a stimulus parameter (e.g., flicker frequency, source intensity, combinations thereof, and the like) may be changed (e.g., increased or decreased) until that change is perceivable by an individual. For example, the parameter to be changed (e.g., adjusted, tuned) may be the step rate (e.g., the rate of change of the parameter).

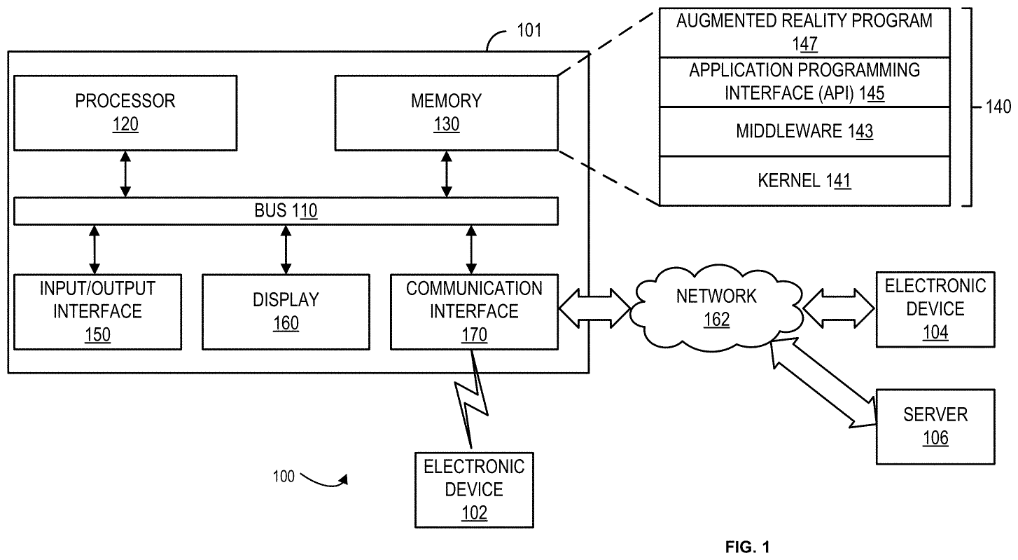

FIG. 1 illustrates a network environment including an electronic device configured for self-administration of a measure of CFF according to various embodiments. Referring to FIG. 1, an electronic device 101 in a network environment 100 is disclosed according to various exemplary embodiments. The electronic device 101 may include a bus 110, a processor 120, a memory 130, an input/output interface 150, a display 160, and a communication interface 170. In a certain exemplary embodiment, the electronic device 101 may omit at least one of the aforementioned constitutional elements or may additionally include other constitutional elements. The electronic device 101 may be, for example, a mobile phone, a tablet computer, a laptop, a desktop computer, a smartwatch, and the like.

The bus 110 may include a circuit for connecting the aforementioned constitutional elements 110 to 170 to each other and for delivering communication (e.g., a control message and/or data) between the aforementioned constitutional elements.

The processor 120 may include one or more of a Central Processing Unit (CPU), an Application Processor (AP), and a Communication Processor (CP). The processor 120 may control, for example, at least one of other constitutional elements of the electronic device 101 and/or may execute an arithmetic operation or data processing for communication. The processing (or controlling) operation of the processor 120 according to various embodiments is described in detail with reference to the following drawings.

The memory 130 may include a volatile and/or non-volatile memory. The memory 130 may store, for example, a command or data related to at least one different constitutional element of the electronic device 101. According to various exemplary embodiments, the memory 130 may store a software and/or a program 140. The program 140 may include, for example, a kernel 141, a middleware 143, an Application Programming Interface (API) 145, and/or an application program (e.g., “application” or “mobile app”) 147, or the like. The application program 147 may be a CFF program, configured for controlling one or more functions of the electronic device 101 and/or an external device (e.g., lighting device). At least one part of the kernel 141, middleware 143, or API 145 may be referred to as an Operating System (OS). The memory 130 may include a computer-readable recording medium having a program recorded therein to perform the method according to various embodiment by the processor 120.

The kernel 141 may control or manage, for example, system resources (e.g., the bus 110, the processor 120, the memory 130, etc.) used to execute an operation or function implemented in other programs (e.g., the middleware 143, the API 145, or the application program 147). Further, the kernel 141 may provide an interface capable of controlling or managing the system resources by accessing individual constitutional elements of the electronic device 101 in the middleware 143, the API 145, or the application program 147.

The middleware 143 may perform, for example, a mediation role so that the API 145 or the application program 147 can communicate with the kernel 141 to exchange data.

Further, the middleware 143 may handle one or more task requests received from the application program 147 according to a priority. For example, the middleware 143 may assign a priority of using the system resources (e.g., the bus 110, the processor 120, or the memory 130) of the electronic device 101 to at least one of the application programs 147. For instance, the middleware 143 may process the one or more task requests according to the priority assigned to the at least one of the application programs, and thus may perform scheduling or load balancing on the one or more task requests.

The API 145 may include at least one interface or function (e.g., instruction), for example, for file control, window control, video processing, or character control, as an interface capable of controlling a function provided by the application 147 in the kernel 141 or the middleware 143.

For example, the input/output interface 150 may play a role of an interface for delivering an instruction or data input from a user or a different external device(s) to the different constitutional elements of the electronic device 101. Further, the input/output interface 150 may output an instruction or data received from the different constitutional element(s) of the electronic device 101 to the different external device(s).

The display 160 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body.

In an embodiment, the display 160 may be configured for emitting light at one or more frequencies (e.g., flicker frequencies). The display 160 may be configured for emitting light at a flicker frequency ranging from about 10 Hz to about 60 Hz. The display 160 may be configured for increasing or decreasing the flicker frequency at which light is emitted. The display 160 may be configured for increasing or decreasing the flicker frequency at which the light is emitted according to a step rate. The step rate may range, for example, from 0.1 Hz/second to 1 Hz/second. In an embodiment, the step rate may be 0.5 Hz/second. The display 160 may also be configured for emitting light at one or more intensities. The one or more intensities may be, for example, from about 2 lux to about 145 lux. In an embodiment, the intensity may be 4 lux. The various intensities may be generated by, for example, varying the value of a resister associated with an LED. The application program 147 may be configured to control the flicker frequency, the step rate, and/or the intensity.

The communication interface 170 may establish, for example, communication between the electronic device 101 and the external device (e.g., electronic device 102, electronic device 104, or a server 106). For example, the communication interface 170 may communicate with the external device (e.g., the second external electronic device 104 or the server 106) via a network 162. The network 162 may make use of both wireless and wired communication protocols.

For example, as a wireless communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), other cellular technologies, combinations thereof, and the like. Further, the wireless communication may include, for example, a near-distance communication protocol 164. The near-distance communication protocol 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

Each of the electronic device 102 and the electronic device 104 may be the same type or different type of the electronic device 101. In an embodiment, the electronic device 102 may be a lighting device. The lighting device may comprise one or more light emitting diodes (LED), one or more liquid crystal displays (LCD), one or more Cold Cathode Fluorescent Lamps (CCFL), combinations thereof, and the like. The lighting device may be configured for emitting light at one or more frequencies (e.g., flicker frequencies). The lighting device may be configured for emitting light at a flicker frequency ranging from about 10 Hz to about 60 Hz. The lighting device may be configured for increasing or decreasing the flicker frequency at which light is emitted. The lighting device may be configured for increasing or decreasing the flicker frequency at which light is emitted according to a step rate. The step rate may range, for example, from 0.1 Hz/second to 1 Hz/second. In an embodiment, the step rate may be 0.5 Hz/second. The lighting device may also be configured for emitting light at one or more intensities. The one or more intensities may be, for example, from about 2 lux to about 145 lux. In an embodiment, the intensity may be 4 lux. The application program 147 may be configured to communicate with the electronic device 102 via the network 164 to control the flicker frequency, the step rate, and/or the intensity.

According to one exemplary embodiment, the server 106 may include a group of one or more servers. According to various exemplary embodiments, all or some of the operations executed by the electronic device 101 may be executed in a different one or a plurality of electronic devices (e.g., the electronic device 102, the electronic device 104, or the server 106). According to one exemplary embodiment, if the electronic device 101 needs to perform a certain function or service either automatically or at a request, the electronic device 101 may request at least some parts of functions related thereto alternatively or additionally to a different electronic device (e.g., the electronic device 102, the electronic device 104, or the server 106) instead of executing the function or the service autonomously. The different electronic device (e.g., the electronic device 102, the electronic device 104, or the server 106) may execute the requested function or additional function and may deliver a result thereof to the electronic device 101. The electronic device 101 may provide the requested function or service either directly or by additionally processing the received result. For this, for example, a cloud computing, distributed computing, or client-server computing technique may be used.

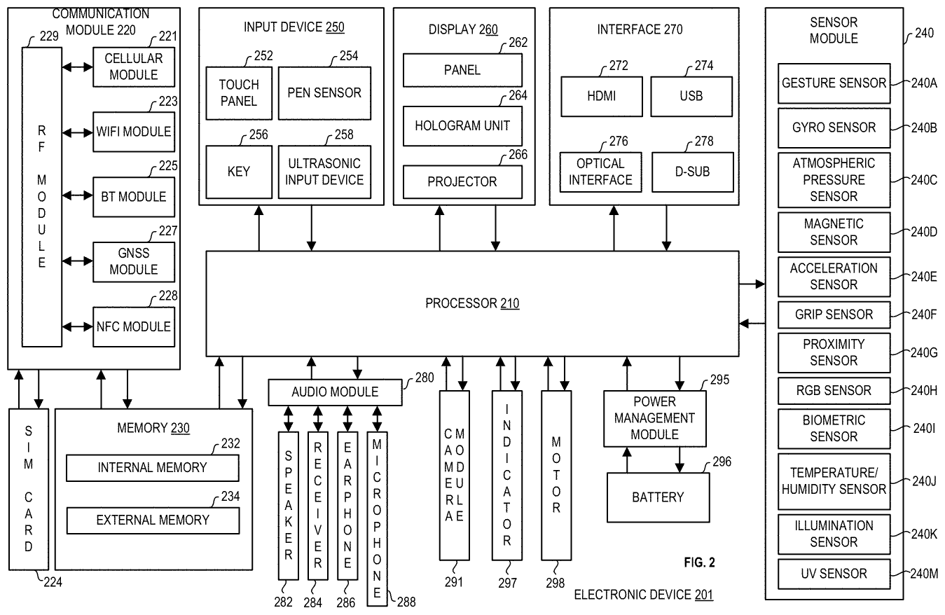

FIG. 2 is a block diagram of an electronic device 201 according to various exemplary embodiments. The electronic device 201 may include, for example, all or some parts of the electronic device 101, the electronic device 102, or the electronic device 104 of FIG. 1. The electronic device 201 may include one or more processors (e.g., Application Processors (APs)) 210, a communication module 220, a subscriber identity module 224, a memory 230, a sensor module 240, an input unit 250, a display 260, an interface 270, an audio module 280, a camera unit 291, a power management module 295, a battery 296, an indicator 297, and a motor 298.

The processor 210 may control a plurality of hardware or software constitutional elements connected to the processor 210 by driving, for example, an operating system or an application program, and may process a variety of data, including multimedia data and may perform an arithmetic operation. The processor 210 may be implemented, for example, with a System on Chip (SoC). According to one exemplary embodiment, the processor 210 may further include a Graphic Processing Unit (GPU) and/or an Image Signal Processor (ISP). The processor 210 may include at least one part (e.g., a cellular module 221) of the aforementioned constitutional elements of FIG. 1. The processor 210 may process an instruction or data, which is received from at least one of different constitutional elements (e.g., a non-volatile memory), by loading it to a volatile memory and may store a variety of data in the non-volatile memory.

The communication module 220 may have a structure the same as or similar to the communication interface 170 of FIG. 1. The communication module 220 may include, for example, the cellular module 221, a Wi-Fi module 223, a BlueTooth (BT) module 225, a GNSS module 227 (e.g., a GPS module, a Glonass module, a Beidou module, or a Galileo module), a Near Field Communication (NFC) module 228, and a Radio Frequency (RF) module 229.

The cellular module 221 may provide a voice call, a video call, a text service, an internet service, or the like, for example, through a communication network. According to one exemplary embodiment, the cellular module 221 may identify and authenticate the electronic device 201 in the communication network by using the subscriber identity module (e.g., a Subscriber Identity Module (SIM) card) 224. According to one exemplary embodiment, the cellular module 221 may perform at least some functions that can be provided by the processor 210. According to one exemplary embodiment, the cellular module 221 may include a Communication Processor (CP).

Each of the WiFi module 223, the BT module 225, the GNSS module 227, or the NFC module 228 may include, for example, a processor for processing data transmitted/received via a corresponding module. According to a certain exemplary embodiment, at least one of the cellular module 221, the WiFi module 223, the BT module 225, the GPS module 227, and the NFC module 228 may be included in one Integrated Chip (IC) or IC package.

The RF module 229 may transmit/receive, for example, a communication signal (e.g., a Radio Frequency (RF) signal). The RF module 229 may include, for example, a transceiver, a Power Amp Module (PAM), a frequency filter, a Low Noise Amplifier (LNA), an antenna, or the like. According to another exemplary embodiment, at least one of the cellular module 221, the WiFi module 223, the BT module 225, the GPS module 227, and the NFC module 228 may transmit/receive an RF signal via a separate RF module.

The subscriber identity module 224 may include, for example, a card including the subscriber identity module and/or an embedded SIM, and may include unique identification information (e.g., an Integrated Circuit Card IDentifier (ICCID)) or subscriber information (e.g., an International Mobile Subscriber Identity (IMSI)).

The memory 230 (e.g., the memory 130) may include, for example, an internal memory 232 or an external memory 234. The internal memory 232 may include, for example, at least one of a volatile memory (e.g., a Dynamic RAM (DRAM), a Static RAM (SRAM), a Synchronous Dynamic RAM (SDRAM), etc.) and a non-volatile memory (e.g., a One Time Programmable ROM (OTPROM), a Programmable ROM (PROM), an Erasable and Programmable ROM (EPROM), an Electrically Erasable and Programmable ROM (EEPROM), a mask ROM, a flash ROM, a flash memory (e.g., a NAND flash memory, a NOR flash memory, etc.), a hard drive, or a Solid State Drive (SSD)).

The external memory 234 may further include a flash drive, for example, Compact Flash (CF), Secure Digital (SD), Micro Secure Digital (Micro-SD), Mini Secure digital (Mini-SD), extreme Digital (xD), memory stick, or the like. The external memory 234 may be operatively and/or physically connected to the electronic device 201 via various interfaces.

The sensor module 240 may measure, for example, a physical quantity or detect an operational status of the electronic device 201, and may convert the measured or detected information into an electric signal. The sensor module 240 may include, for example, at least one of a gesture sensor 240A, a gyro sensor 240B, a pressure sensor 240C, a magnetic sensor 240D, an acceleration sensor 240E, a grip sensor 240F, a proximity sensor 240G, a color sensor 240H (e.g., a Red, Green, Blue (RGB) sensor), a bio sensor 240I, a temperature/humidity sensor 240J, an illumination sensor 240K, an Ultra Violet (UV) sensor 240M, an ultrasonic sensor 240N, and an optical sensor 240P. According to one exemplary embodiment, the optical sensor 240P may detect ambient light and/or light reflected by an external object (e.g., a user's finger. etc.), and convert the detected ambient light into a specific wavelength band by means of a light converting member. For example, the illumination sensor 240K may comprise a light meter sensor. An exemplary sensor may be the Amprobe LM-200 LED, however any suitable light meter sensor may be used. In an embodiment, the illumination sensor 240K may be pressed against a diffuser of the lighting device. Additionally or alternatively, the sensor module 240 may include, for example, an E-nose sensor, an ElectroMyoGraphy (EMG) sensor, an ElectroEncephaloGram (EEG) sensor, an ElectroCardioGram (ECG) sensor, an Infrared (IR) sensor, an iris sensor, and/or a fingerprint sensor. The sensor module 240 may further include a control circuit for controlling at least one or more sensors included therein. In a certain exemplary embodiment, the electronic device 201 may further include a processor configured to control the sensor module 204 either separately or as one part of the processor 210, and may control the sensor module 240 while the processor 210 is in a sleep state.

The input device 250 may include, for example, a touch panel 252, a (digital) pen sensor 254, a key 256, or an ultrasonic input device 258. The touch panel 252 may recognize a touch input, for example, by using at least one of an electrostatic type, a pressure-sensitive type, and an ultrasonic type detector. In addition, the touch panel 252 may further include a control circuit. The touch penal 252 may further include a tactile layer and thus may provide the user with a tactile reaction (e.g., haptic feedback). For instance, the haptic feedback may be associated with the frequency of the emitted light. The haptic feedback may be associated with the user input.

The (digital) pen sensor 254 may be, for example, one part of a touch panel, or may include an additional sheet for recognition. The key 256 may be, for example, a physical button, an optical key, a keypad, or a touch key. The ultrasonic input device 258 may detect an ultrasonic wave generated from an input means through a microphone (e.g., a microphone 288) to confirm data corresponding to the detected ultrasonic wave.

The display 260 (e.g., the display 160) may include a panel 262, a hologram unit 264, or a projector 266. The panel 262 may include a structure the same as or similar to the display 160 of FIG. 1. The panel 262 may be implemented, for example, in a flexible, transparent, or wearable manner. The panel 262 may be constructed as one module with the touch panel 252. According to one exemplary embodiment, the panel 262 may include a pressure sensor (or a force sensor) capable of measuring a pressure of a user's touch. The pressure sensor may be implemented in an integral form with respect to the touch panel 252, or may be implemented as one or more sensors separated from the touch panel 252.

The hologram unit 264 may use an interference of light and show a stereoscopic image in the air. The projector 266 may display an image by projecting a light beam onto a screen. The screen may be located, for example, inside or outside the electronic device 201. According to one exemplary embodiment, the display 260 may further include a control circuit for controlling the panel 262, the hologram unit 264, or the projector 266.

The interface 270 may include, for example, a High-Definition Multimedia Interface (HDMI) 272, a Universal Serial Bus (USB) 274, an optical communication interface 276, or a D-subminiature (D-sub) 278. The interface 270 may be included, for example, in the communication interface 170 of FIG. 1. Additionally or alternatively, the interface 270 may include, for example, a Mobile High-definition Link (MHL) interface, a Secure Digital (SD)/Multi-Media Card (MMC) interface, or an Infrared Data Association (IrDA) standard interface.

The audio module 280 may bilaterally convert, for example, a sound and electric signal. At least some constitutional elements of the audio module 280 may be included in, for example, the input/output interface 150 of FIG. 1. The audio module 280 may convert sound information, which is input or output, for example, through a speaker 282, a receiver 284, an earphone 286, the microphone 288, or the like.

The camera module 291 may comprise, for example, a device for image and video capturing, and according to one exemplary embodiment, may include one or more image sensors (e.g., a front sensor or a rear sensor), a lens, an Image Signal Processor (ISP), or a flash (e.g., LED or xenon lamp).

The power management module 295 may manage, for example, power (e.g., consumption or output) of the electronic device 201. According to one exemplary embodiment, the power management module 295 may include a Power Management Integrated Circuit (PMIC), a charger Integrated Circuit (IC), or a battery fuel gauge. The PMIC may have a wired and/or wireless charging type. The wireless charging type may include, for example, a magnetic resonance type, a magnetic induction type, an electromagnetic type, or the like, and may further include an additional circuit for wireless charging, for example, a coil loop, a resonant circuit, a rectifier, or the like. A battery gauge may measure, for example, residual quantity of the battery 296 and voltage, current, and temperature during charging. The battery 296 may include, for example, a non-rechargeable battery, a rechargeable batter, and/or a solar battery.

The indicator 297 may display a specific state, for example, a booting state, a message state, a charging state, or the like, of the electronic device 201 or one part thereof (e.g., the processor 210). The motor 298 may convert an electric signal into a mechanical vibration, and may generate a vibration or haptic effect. Although not shown, the electronic device 201 may include a processing device (e.g., a GPU) for supporting a mobile TV. The processing device for supporting the mobile TV may process media data conforming to a protocol of, for example, Digital Multimedia Broadcasting (DMB), Digital Video Broadcasting (DVB), MediaFlo™, or the like.

Each of the constitutional elements described in the present document may consist of one or more components, and names thereof may vary depending on a type of an electronic device. The electronic device, according to various exemplary embodiments, may include at least one of the constitutional elements described in the present document. Some of the constitutional elements may be omitted, or additional other constitutional elements may be further included. Further, some of the constitutional elements of the electronic device, according to various exemplary embodiments, may be combined and constructed as one entity so as to equally perform functions of corresponding constitutional elements before combination.

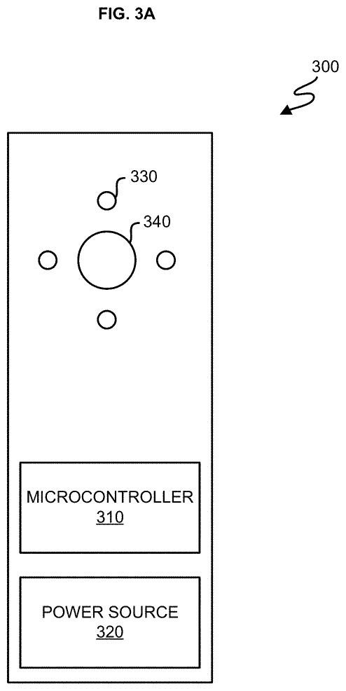

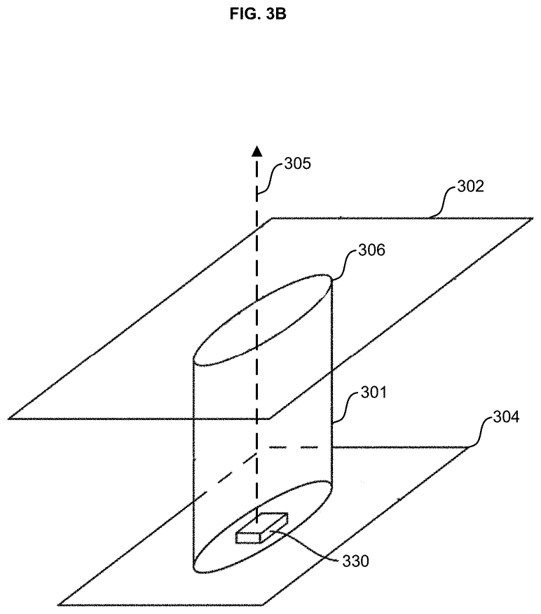

FIG. 3A illustrates a lighting device 300 according to various embodiments of the present disclosure. The lighting device 300 may comprise a microcontroller 310, a power source 320, one or more light sources 330, and one or more light sources 340. In one embodiment, the microcontroller 310 may include and/or be in communication with, an analog emitter source driver, such as an LED driver, to selectively provide power to the one or more light sources 330 and/or the one or more light sources 340. In an embodiment, the one or more light sources 330 may form an LED array. The microcontroller 310 may selectively provide power to the LED array. In one non-limiting example, the analog emitter source driver may include a low noise analog LED driver as one or more adjustable current sources to selectively set and/or adjust (e.g., vary) emitted light intensity level and/or frequency (e.g., flicker frequency). The microcontroller 310 may also communicate with a memory, or other onboard storage device configured for storing and reading data. The light intensity level may be adjusted according to a measurement of ambient light (e.g., according to the illumination sensor 409 (as described further herein). The more ambient light is detected, the greater the emitted light intensity level.

In one embodiment, the microcontroller 310 may be configured to transmit and/or receive data via a wireless network interface to and/or from an external device (e.g., the electronic device 101). The microcontroller may comprise the wireless network interface. The wireless network interface may be a Bluetooth connection, an antenna, or other suitable interface. In one embodiment, the wireless network interface is a Bluetooth Low Energy (BLE) module. In one non-limiting example, the wireless network interface and the microcontroller 310 are integrated in one unitary component, such as an RFduino microcontroller with built-in BLE module, a Nordic Semiconductor microcontroller, or a Cypress microcontroller with BLE module. The RFduino may drive square waves at a duty cycle (e.g., a 50% duty cycle) such that a pulse remains high during half a period and low during the remaining half. The RFduino may drive frequencies ranging from around 0 Hz to around 100 Hz. The RFduino may drive the frequencies at a step rate, for example, a step rate of 0.5 Hz/sec (e.g., 0.1 Hz/0.2 sec).

The one or more light sources 330 and one or more light sources 340 may comprise one or more LEDs. The one or more light sources 330 may be configured to assist in aligning the lighting device 300 to a user's vision in order to measure CFF. The one or more light sources 330 may be recessed within a housing of the lighting device 300. The one or more light sources 340 may be configured to emit light at varying frequencies and/or intensities in order to measure CFF. Further, any of the sensors described herein may be used to alight the lighting device 300 to a user's vision. For example, the gyro sensor may determine a vertical or horizontal orientation relative to the ground. Upon determining that the lighting device 300 is orientated approximately perpendicular to the ground, the lighting device 300 may indicate to the user that the lighting device 300 is oriented as such. For example, the one or more light sources 330 may indicate the orientation by, for example, blinking, or changing color or intensity. The lighting device 300 may send a message to the device comprising the user interface element wherein the message indicates the orientation of the lighting device 300. For example, one or more audio tones or visual cues may indicate to the user that the lighting device 300 is properly aligned for user. The one or more light sources 340 may comprise a wide range LED technologies of carious luminous intensities. For example, the one or more light sources 330 or 340 may comprise a C503D-WAN-CCBEB151 LED with luminous intensities from 28 cd to 64 cd, paired with a milky white diffuser in front of the one or more light sources 340.

FIG. 3B shows a simplified perspective view of an illustrative light source recess 301 configured for constraining both vertical and horizontal directions of light emitted from the light source 330. The light source recess 301 may travel from an exterior housing 302 to an internal mounting surface 304. The light source 330 may be mounted on the internal mounting surface 304. The light source recess 301 may be configured such that light emitted by the light source 330 travels in a specific direction 305 when exiting an opening 306. The direction 305 may be configured to, in conjunction with light existing multiple other openings 306 in the lighting device 300, focus light such that a user of the lighting device 300 will only see all light emitted from all light sources 330 when the lighting device 300 is properly aligned to the user's vision.

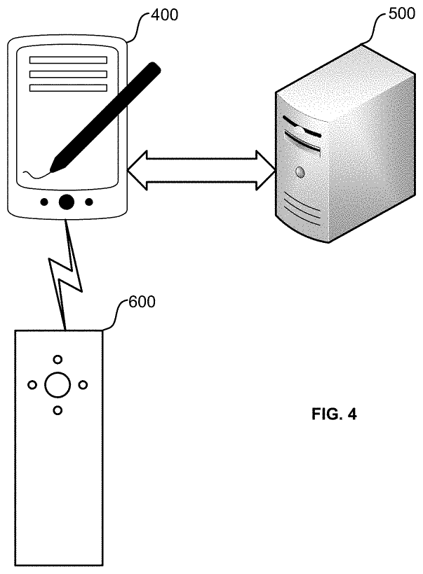

FIG. 4 illustrates a system according to various embodiments of the present disclosure that may include a first electronic device 400, a lighting device 600, and a second electronic device 500. According to various embodiments, the first electronic device 400 may be connected (e.g., paired) with the lighting device 600 through a first communication link (for example, wired communication or wireless communication) and connected with the second electronic device 500 through second communication link (for example, wired communication or wireless communication). According to various embodiments, the first communication link may include a wired communication scheme such as cable communication or a short-range wireless communication scheme such as BT, BLE, or Near-Field Magnetic Induction (NFMI). According to various embodiments, the first communication link is not limited thereto and may include various wireless communication techniques such as, for example, Wi-Fi, NFC, ZigBee, UWB, or IrDA. According to various embodiments, the second communication link may include a mobile communication scheme such as cellular communication or a wireless communication scheme such as Wi-Fi.

According to various embodiments, the first electronic device 400 may initiate a CFF measurement by communicating with the connected lighting device 600 to cause the connected lighting device 600 to emit light at a frequency. The first electronic device 400 may cause the connected lighting device 600 to vary the frequency at which the light is emitted according to a step rate. Once a user determines that the emitted light has “fused,” the user may interact with the first electronic device 400 to indicate the fusion. The first electronic device 400 may log one or more of a time and/or date of the indication and a frequency at which the light was emitted at the time and/or date. The first electronic device 400 may repeat the process and log the results. In various embodiments, the first electronic device 400 may cause the connected lighting device 600 to emit light at a first frequency and increase the first frequency until receiving a first indication of fusion. For example, the first frequency may be 25.0 Hz and the first frequency may be increased at a step rate of 0.5 Hz/sec. The first electronic device 400 may then cause the connected lighting device 600 to emit light at a second frequency (higher than the first frequency) and decrease the second frequency until receiving a second indication of fusion. For example, the second frequency may be 55.0 Hz and the second frequency may be decreased at a step rate of 0.5 Hz/sec. The mathematical average of the frequencies corresponding to the first indication and the second indication may be used to determine a CFF value of a current measurement (e.g., a CFF measurement). The process may be repeated from a third frequency, a fourth frequency, a fifth frequency, etc. until a number of tests have been performed. An average of all tests may define a user's CFF measurement.

According to various embodiments, the first electronic device 400 may transmit data indicative of the CFF measurement, the indication(s), the time(s) and/or date(s), and the like, to the second electronic device 500 (e.g., a remote server). According to various embodiments, the second electronic device 500 may be connected to the first electronic device 400 through wireless communication and may receive data from the first electronic device 400 in real time. According to various embodiments, the second electronic device 500 may display various UIs or GUIs based at least partially on the received data.

According to various embodiments, the first electronic device 400 may include, for example, a smartphone, tablet, Personal Digital Assistant (PDA), a tablet, a Personal Computer (PC), combinations thereof, and the like. According to various embodiments, the first electronic device 400 may display various User Interfaces (UIs) or Graphical User Interfaces (GUIs) related to using the lighting device 600. The operation and relevant screen examples of the first electronic device 400 according to various embodiments will be described in detail with reference to the figures below.

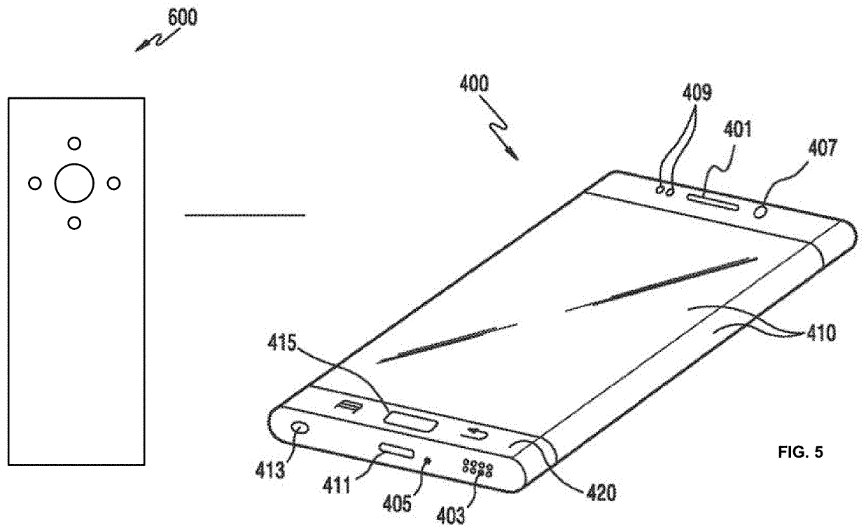

FIG. 5 illustrates the electronic device 400 and the lighting device 600 according to various embodiments of the present disclosure. According to various embodiments, the electronic device 400 may include a display 410, a housing (or a body) 420 to which the display 410 is coupled while the display 410 is seated therein, and an additional device formed on the housing 420 to perform the function of the electronic device 400. According to various embodiments, the additional device may include a first speaker 401, a second speaker 403, a microphone 405, sensors (for example, a front camera module 407 and an illumination sensor 409), communication interfaces (for example, a charging or data input/output port 411 and an audio input/output port 413), and a button 415. According to various embodiments, when the electronic device 400 and the lighting device 600 are connected through a wired communication scheme, the electronic device 400 and the lighting device 600 may be connected based on at least some ports (for example, the data input/output port 411) of the communication interfaces.

According to various embodiments, the display 410 may include a flat display or a bended display (or a curved display) which can be folded or bent through a paper-thin or flexible substrate without damage. The bended display may be coupled to the housing 420 to remain in a bent form. According to various embodiments, the electronic device 400 may be implemented as a display device, which can be quite freely folded and unfolded such as a flexible display, including the bended display. According to various embodiments, in a Liquid Crystal Display (LCD), a Light Emitting Diode (LED) display, an Organic LED (OLED) display, or an Active Matrix OLED (AMOLED) display, the display 410 may replace a glass substrate surrounding liquid crystal with a plastic film to assign flexibility to be folded and unfolded. The display can be used to run the protocol to measure CFF (i.e., the method of limits), turn the calibration lights on or off, and view results. To start the measurement and record input, a person can press anywhere on the display (i.e., a person does not look at the screen while measuring CFF).

According to various embodiments, the electronic device 400 may be connected to the lighting device 600. According to various embodiments, the electronic device 400 may be connected to the lighting device 600 based on wireless communication (for example, Bluetooth or Bluetooth Low Energy (BLE)).

According to various embodiments, the electronic device 400 may be connected to the lighting device 600, and may generate relevant data (for example, measurements of CFF, including historical measurements) for monitoring and/or diagnosis of disease state and transmit the generated data to the second electronic device 500.

According to various embodiments, the electronic device 400 may process an operation related to starting a measurement of CFF (for example, acquire one or more indications from a user by controlling the lighting device 600) using the lighting device 600 and displaying and/or transmitting a result to the second electronic device 500. In an embodiment, the electronic device 400 can send an instruction to the lighting device 600 to cause the lighting device 600 to emit light. In an embodiment, the instruction can cause the lighting device 600 to emit light according to a pre-programmed pattern (e.g., frequency, intensity, step rate) stored on the lighting device 600. In another embodiment, the instruction can indicate a frequency at which to begin emitting the light (e.g., flickering light). The instruction can indicate a step rate at which to vary the frequency (e.g., increase or decrease the frequency). A user, observing the light emitted from the lighting device 600, may indicate via a touchscreen, button, and the like, of the electronic device 400 when the user perceives that the flickering emitted light has fused into a single emission and is no longer flickering. The frequency at which the light was emitted when the user made the indication may be logged by the electronic device 400. The instruction may indicate that the lighting device 600 is to repeat the light emission (starting at the same or a different frequency), and another indication may be received from the user, indicating that the flickering emitted light has fused into a single emission and is no longer flickering. Again, the frequency at which the light was emitted when the user made the indication may be logged by the electronic device 400. The average of the frequencies may be determined as a measurement of CFF. The measurement of CFF may be used to determine a disease state. For example, the measurement of CFF may indicate a diagnosis of minimal hepatic encephalopathy (MHE) or other diseases. The measurement of CFF may be added to a user profile as part of a historical record of CFF measurements for a user.

According to various embodiments, the electronic device 400 may receive lighting control information from the second electronic device 500 and perform various operations (for example, configure one or more frequencies, step rates, and/or intensities).

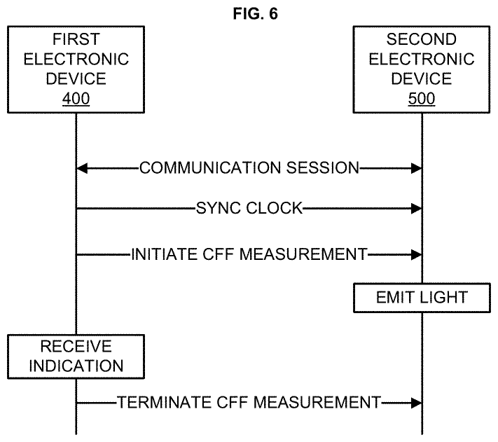

FIG. 6 illustrates a CFF measurement process according to various embodiments of the present disclosure. The first electronic device 400 (e.g., a smartphone) may open a communication session with the second electronic device 500 (e.g., a lighting device). Optionally, the first electronic device 400 may send an instruction to the second electronic device 500 to sync internal clocks of both devices. The first electronic device 400 may send an instruction to the second electronic device 500 to cause the second electronic device 500 to initiate a CFF measurement process. In various embodiments, the instruction may cause light to be emitted from, for example, one or more of the one or more light sources 330 and/or the one or more light sources 340. The instruction may comprise one or more frequencies, one or more step rates, and one or more intensities associated with the emitted light. The second electronic device 500 may receive the instruction. The second electronic device 500 may, based on the instruction, emit light at a frequency (e.g., flicker frequency) and intensity, and vary the frequency according to the one or more step rates. The first electronic device 400 may receive an indication from a user. The indication may be received via the components described herein such as, for example, the touchscreen, the key, combinations thereof, and the like. A time may be associated with the indication. For example, the indication may reflect a time when the user perceives the emitted light as being fused and no longer flickering. The first electronic device 400 may, based on the synced clocks, determine the frequency associated with the time of the received indication. For example, the first electronic device 400 may query the second electronic device 500 for the frequency associated with the time of the received indication. The first electronic device 400 may cause the CFF measurement process to be repeated any number of times. Finally, the first electronic device 400 may terminate the CFF measurement process.

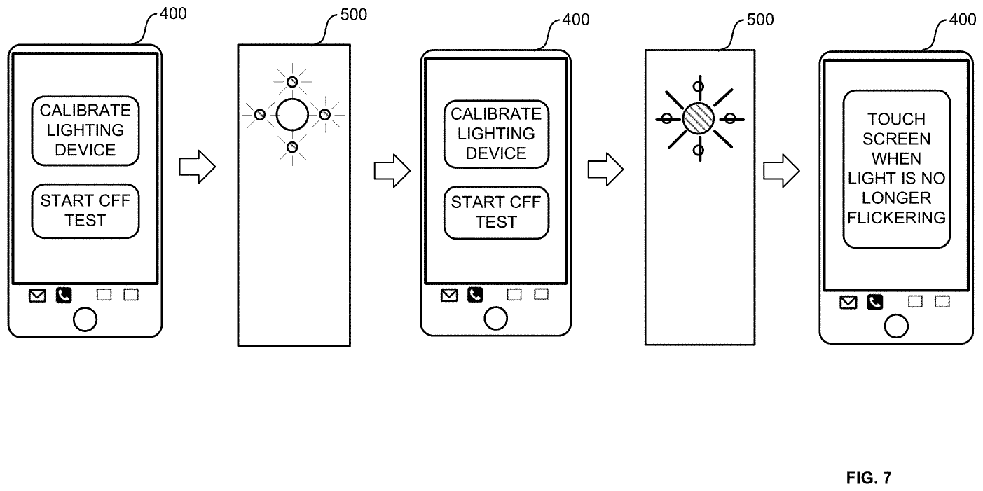

FIG. 7 illustrates a CFF measurement process according to various embodiments of the present disclosure. A user may launch a CFF application (e.g., software program) resident on the first electronic device 400. The CFF application may initiate a communication session with the second electronic device 500. The user may engage a user interface element on the first electronic device 400 to calibrate the second electronic device 500. In response, the second electronic device 500 may activate one or more light sources (e.g., the light sources 330) to enable the user to align the user's vision to the second electronic device 500. For example, the one or more light sources 330 may be recessed (as described above) such that the user may only view the light when the recess is level with the eyes of the user (e.g., the viewing angle is around 0 degrees). The user may engage the user interface element on the first electronic device 400 to start a CFF measurement process (e.g., CFF Test). In response, the second electronic device 500 may activate the one or more light sources to emit light at a frequency (e.g., flicker frequency) and intensity. The second electronic device 500 may vary the frequency (e.g., increase or decrease) at a step rate until the user engages the user interface element on the first electronic device 400. The user may engage the user interface element to indicate that the user perceived the flickering light as the fused light. In an embodiment, the first electronic device 400 may guide the user through the CFF measurement process via voice or other audio-based prompts. The first electronic device 400 may guide the user through the CFF measurement through text or other visual-based prompts.

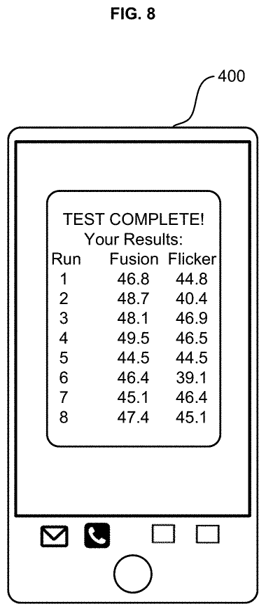

As seen in FIG. 8, results from each iteration of the CFF measurement process may be displayed to the user on the first electronic device 400. In an embodiment, a user with limited dexterity or hand tremors might have unintended inputs due to accidentally pressing the screen of the first electronic device 400 in rapid succession. The first electronic device 400 may be configured to introduce a 2-second delay between presses during which the screen remains inactive. This may prevent further misreports by such a user.

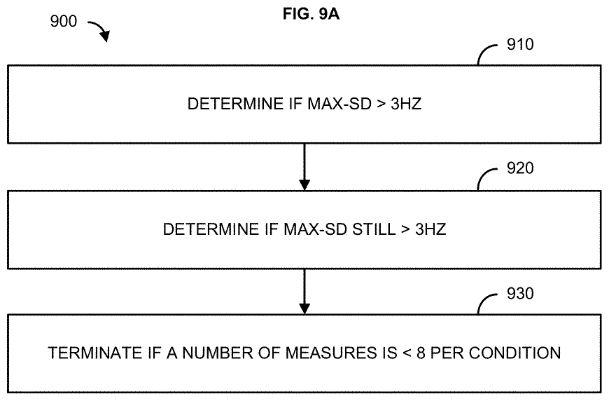

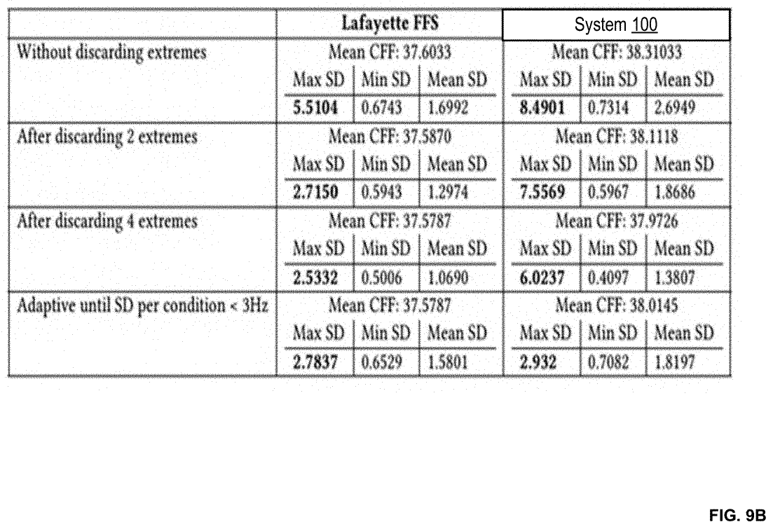

FIG. 9A shows an adaptive algorithm 900 for obtaining accurate CFF measurement results. The adaptive algorithm 900 may be applied to identify and remove outliers. The adaptive algorithm may comprise various steps. For example, at 910, it may be determined whether a maximum standard deviation exceeds 3 Hz (e.g., max-sd>3 Hz). If at 910, it is determined that max-sd>3 Hz then the adaptive algorithm 900 may identify two extreme CFF measurements. The two extreme CFF measurements may comprise a lowest CFF measurement and a highest CFF measurement. The lowest CFF measurement and the highest CFF measurement may be removed from consideration (e.g., “discarded). The adaptive algorithm may comprise, for example, at 920, determining if max-sd is still >3 Hz. If at 910, it is determined that max-sd is still >3 Hz then the adaptive algorithm 900 may repeat step 910. The adaptive algorithm may comprise, for example, at 930, terminating if a number of measures is <8 per condition. This process may be repeated.

FIG. 9B shows an example table of descriptive statistics. Descriptive statistics for the Adaptive Algorithm used in the Comparative study data cleaning for each device (in Hz). The goal of the algorithm is to reduce maximum standard deviation while having minimal impact on the mean CFF. As seen here, using the adaptive algorithm Beacon achieved a maximum standard deviation of 2.93 Hz compared to 2.78 Hz achieved by Lafayette FFS. The mean CFF remains unaffected with a difference of only 0.29 Hz in Beacon when not using and using the algorithm and 0.02 Hz in Lafayette FFS.

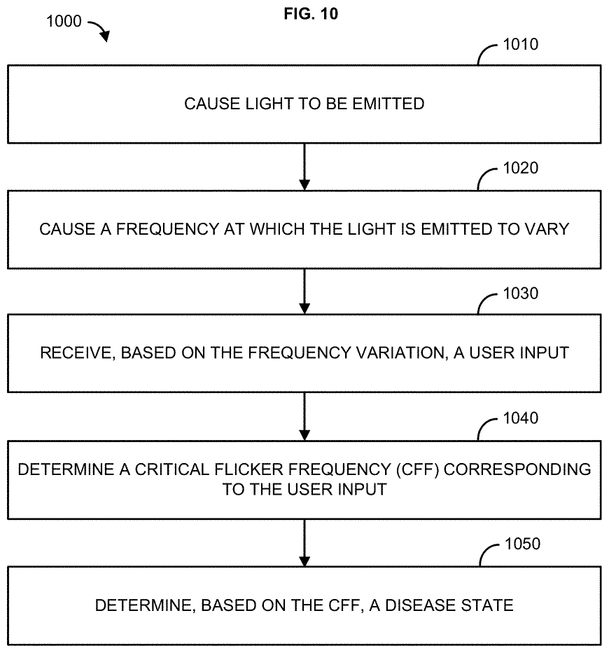

FIG. 10 shows an example method 1000. The method 1000 may be implemented by any suitable computing device such as the computing device 101, the electronic device 102, the electronic device 104, the electronic device 201, the lighting device 300, or any other devices described herein.

At 1010, light may be caused to be emitted. Causing the light to be emitted may comprise sending a command to a lighting device (e.g., the lighting device). The command may comprise data associated with the light to be emitted. For example, the data associated with the light to be emitted may comprise a color, an intensity, a frequency at which the light should be intermittently emitted (e.g., a flicker frequency), combinations thereof, and the like. The data may be sent from a device such an electronic device (e.g., the electronic device 102 or the electronic device 104). The data may be received by, for example, the lighting device. The data may cause the lighting device to emit the light. For example, the lighting device may comprise at least one light source. For example, the at least one light source may be the one or more light sources 330 and/or the one or more light sources 340. Causing light to be admitted may comprise causing the light to be intermittently emitted at a first frequency (e.g., flicker frequency). For example, the microcontroller 310 may include and/or be in communication with, an analog emitter source driver, such as an LED driver, to selectively provide power to the one or more light sources 330 and/or the one or more light sources 340. In an embodiment, the one or more light sources 330 may form an LED array. The microcontroller 310 may selectively provide power to the LED array. In one non-limiting example, the analog emitter source driver may include a low noise analog LED driver as one or more adjustable current sources to selectively set and/or adjust (e.g., vary) emitted light intensity level and/or frequency (e.g., flicker frequency).

At 1020, the frequency at which the light is emitted may be caused to vary. Causing the light to be emitted may comprise causing the light to be emitted at a first frequency and wherein causing the frequency at which the light is emitted to vary comprises increasing the first frequency to a second frequency over a first time period. Causing the light to be emitted may comprise causing the light to be emitted at a third frequency and wherein causing the frequency at which the light is emitted to vary comprises decreasing the third frequency to a fourth frequency over a second time period. For example, the microcontroller 310 may be integrated in one unitary component, such as an RFduino microcontroller with built-in BLE module, a Nordic Semiconductor microcontroller, or a Cypress microcontroller with BLE module. The RFduino may drive square waves at a duty cycle (e.g., a 50% duty cycle) such that a pulse remains high during half a period and low during the remaining half. The RFduino may drive frequencies ranging from around 0 Hz to around 100 Hz. The RFduino may drive the frequencies at a step rate, for example, a step rate of 0.5 Hz/sec (e.g., 0.1 Hz/0.2 sec).

At 1030, a user input may be received. For example, a user may engage the user interface element on the first electronic device 400 to start a CFF measurement process (e.g., CFF Test). In response, the second electronic device 500 may activate the one or more light sources 330 or 340 to emit light at a frequency (e.g., flicker frequency) and intensity. The second electronic device 500 may vary the frequency (e.g., increase or decrease) at a step rate until the user engages the user interface element on the first electronic device 400. The user may engage the user interface element to indicate that the user perceived the flickering light as the fused light. In an embodiment, the first electronic device 400 may guide the user through the CFF measurement process via voice or other audio-based prompts. The first electronic device 400 may guide the user through the CFF measurement through text or other visual-based prompts. Likewise, the first electronic device 400 may guide the user through the CFF measurement through audio or other sound-based prompts.

At 1040, a CFF may be determined. For example, the flicker frequency of a light source may be gradually increased until the viewer indicates (via the user interface element) that the light source is no longer appears as a flickering light source but rather as a steady light source. This frequency may indicate the CFF. The CFF for the user may represent a threshold at which the light is seen half the time as flickering and half the time as fused. For example, a discrimination method may be implemented to determine a CFF threshold. In a similar respect the flicker frequency of the light source may be gradually decreased until the viewer feels that the light source is fused (e.g., not flickering). Likewise, this frequency is also referred to as the CFF threshold. The mathematical average of the flicker frequencies at the two points may be used to represent the CFF value of the measurement (e.g., the CFF measurement).

Measuring CFF may incorporate the appropriate threshold detection algorithm as described here (e.g., with respect to FIGS. 9A-9B). The threshold detection algorithm may implement the method of limits, which focuses on the influence and relationship between stimuli and the sensation and perception of these stimuli by an individual. For example, a stimulus (e.g., light in the case of CFF) is presented and a stimulus parameter (e.g., flicker frequency, source intensity, combinations thereof, and the like) may be changed (e.g., increased or decreased) until that change is perceivable by an individual. For example, the parameter to be changed (e.g, adjusted, tuned) may be the step rate (e.g., the rate of change of the parameter).

At 1050, a disease state may be determined. For example, the disease state may be determined based on the CFF. Determining, based on the CFF, the disease state may comprise determining that the CFF is indicative of minimal hepatic encephalopathy. The method 1000 may further comprise, after receiving the first user input, varying the frequency at which the light is emitted, receiving, based on the frequency variation, a second user input, and wherein determining the disease state is further based on a second CFF. The method 1000 may further comprise, determining an average of the CFF and the second CFF and wherein determining the disease state comprises determining that the average of the CFF and the second CFF is indicative of the disease state. The method 1000 may further comprise, determining that a light source is aligned with a user's vision, prior to causing light to be emitted. The method 1000 may further comprise determining an ambient lighting intensity and adjusting, based on the ambient lighting intensity, the CFF. The method 1000 may further comprise storing, in a user profile, at least one of: the CFF as a portion of a historical record of CFF's, a light emission frequency variation range, an average critical flicker frequency (CFF), and/or an examination schedule.

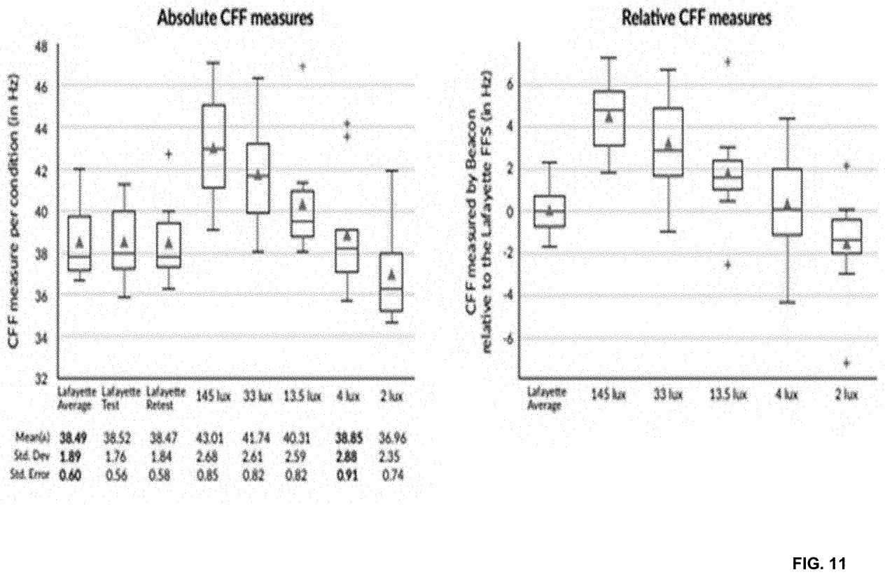

FIG. 11, shows exemplary data. FIG. 11 shows Absolute CFF measures on the left and Relative CFF measures on the right. FIG. 11 indicates a measured CFF may be proportional to the light source intensity. For example, FIG. 11 shows an exemplary case where 145 lux had a mean CFF of 43.01 Hz, and 2 lux had a mean of 36.96 Hz. The difference between the CFF measured using 145 lux and 2 lux intensities is 6.05 Hz, or 15.7% of the average mean (38.49 Hz). The lines in the plot represent the median; the triangles, the mean. The absolute values of the 7 conditions (5 light source intensities, Lafayette test, and Lafayette retest) alongside a new calculated measure called the “Lafayette average” are obtained by combining the test and retest scores. The table underneath the plot shows the corresponding descriptive statistics.

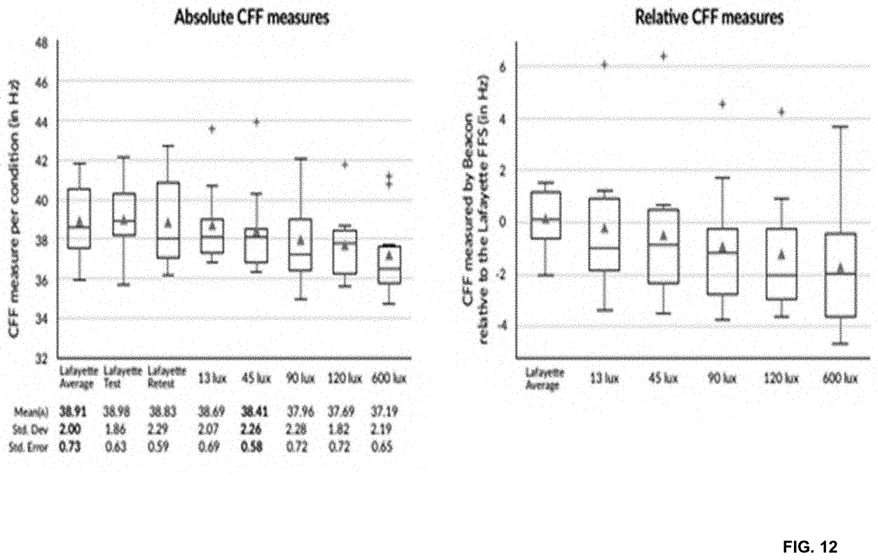

FIG. 12 shows exemplary data. FIG. 12 shows Absolute CFF measures on the left and Relative CFF measures on the right. The horizontal lines in the plot represent the median; the triangles, the mean. FIG. 12 indicates the absolute values of the various conditions (e.g., light source intensities) alongside a new measure average which combines a test and retest score. The plot shows a trend that CFF value is indirectly proportional to ambient light intensity. The table underneath the plot shows the corresponding descriptive statistics. The plot shows the values of ambient light intensities relative to average by using it as the baseline. Ambient light intensity of 45 lux was chosen for the comparative study since it was deemed to be a more easily achievable ambient light setting in clinics and homes.

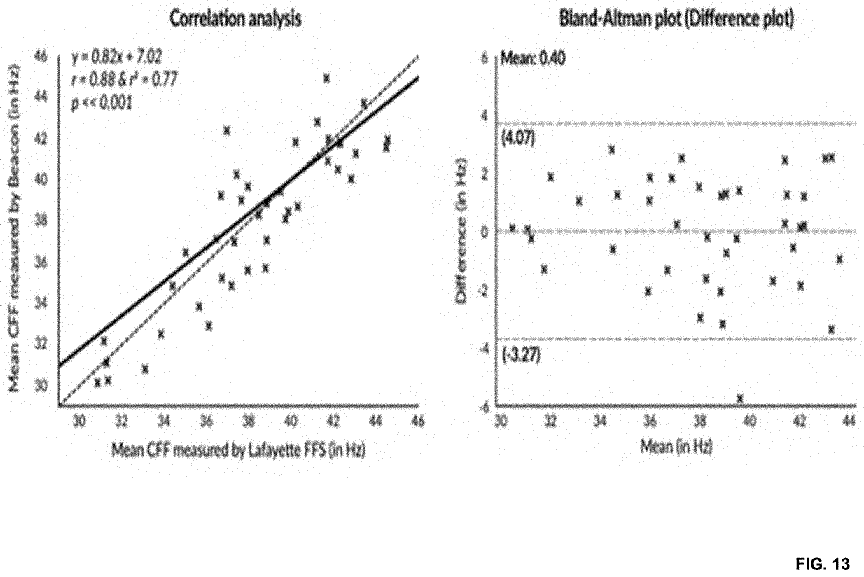

FIG. 13 shows exemplary data. FIG. 13 shows an exemplary correlation analysis on the left and a Bland-Altman plot on the right. Also known as a difference plot, Bland-Altman plot is ideal for comparing two measurement techniques (or devices). The X-axis represents the mean of the CFF measurements for a plurality of devices and the Y-axis represents the absolute difference between the measurements taken by the devices. The plot includes the line for the mean difference between the measurements (0.40 Hz) and the 2 lines showing the 2 s (1.96 standard deviation) limits of differences between the measurements (also called 95% limits of agreement) which span from −3.27 Hz to +4.07 Hz. The limits of agreement may indicate the difference in CFF measured by will be at most ±3.67 Hz for 95% of the measurements. FIG. 13 may indicate a regression analysis shows a strong correlation between the CFF measure by several devices with a Pearson's R of 0.88. Right: The Bland-Altman plot shows the mean difference between measurements to be 0.4 Hz with a maximum difference of at most ±3.67 Hz for 95% of the measurements. Determining the correlation may comprise performing either or both of a Pearson or Spearman correlation analysis.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
causing light to be emitted at a first frequency;
causing the first frequency at which the light is emitted to vary;
receiving, based on the varied first frequency, a user input;
determining a critical flicker frequency (CFF) corresponding to the user input; and
determining, based on the CFF, a disease state.

2. The method of claim 1, wherein causing the light to be emitted comprises sending a command to a light device, wherein the light device emits the light.

3. The method of claim 1, wherein the CFF for a user represents a threshold at which the light is seen half the time as flickering and half the time as fused.

4. The method of claim 1, wherein causing the light to be emitted comprises causing the light to be emitted at the first frequency and wherein causing the first frequency at which the light is emitted to vary comprises increasing the first frequency to a second frequency over a first time period.

5. The method of claim 1, wherein causing the light to be emitted comprises causing the light to be emitted at the first frequency and wherein causing the first frequency at which the light is emitted to vary comprises decreasing the first frequency to a third frequency over a second time period.

6. The method of claim 1, wherein determining, based on the CFF, the disease state comprises determining that the CFF is indicative of minimal hepatic encephalopathy.

7. The method of claim 1, further comprising:
after receiving the user input, varying the first frequency at which the light is emitted;
receiving, based on the varied first frequency, a second user input; and
wherein determining the disease state is further based on a second CFF.

8. The method of claim 7, further comprising:
determining an average of the CFF and the second CFF; and
wherein determining the disease state comprises determining that the average of the CFF and the second CFF is indicative of the disease state.

9. The method of claim 1, further comprising determining that a light source is aligned with a user's vision, prior to causing light to be emitted.

10. The method of claim 1, further comprising:
determining an ambient lighting intensity; and
adjusting, based on the ambient lighting intensity, the CFF.

11. The method of claim 1, further comprising:
storing, in a user profile, at least one of: the CFF as a portion of a historical record of CFF's,
a light emission frequency variation, an average critical flicker frequency (CFF), or an examination schedule.

12. An apparatus comprising:
one or more processors; and
memory storing processor executable instructions that, when executed by the one or more processors, cause the apparatus to:
cause light to be emitted;
cause a first frequency at which the light is emitted to vary;
receive, based on the varied first frequency, a first user input;
determine a critical flicker frequency (CFF) corresponding to the first user input; and
determine, based on the CFF, a disease state.

13. The apparatus of claim 12, wherein the processor executable instructions that, when executed by the one or more processors, cause light to be emitted, further cause light to be emitted by causing a command to be sent to a light device, wherein the light device emits the light.

14. The apparatus of claim 12, wherein the processor executable instructions that, when executed by the one or more processors, cause the apparatus to cause the first frequency at which the light is emitted to vary, cause the first frequency at which the light is emitted to vary by one or more of: increasing the first frequency to a second frequency over a first time period or decreasing the first frequency to a third frequency over a second time period.

15. The apparatus of claim 12, wherein the processor executable instructions when executed by the one or more processors, further cause the apparatus to:
after receiving the first user input, vary the first frequency at which the light is emitted;
receive, based on the varied first frequency, a second user input; and
wherein determining the disease state is further based on a second CFF.

16. The apparatus of claim 15, wherein the processor executable instructions when executed by the one or more processors, further cause the apparatus to:
determine an average of the CFF and the second CFF; and
wherein determining the disease state comprises determining that the average of the CFF and the second CFF is indicative of the disease state.

17. The apparatus of claim 12, wherein the processor executable instructions, when executed by the one or more processors, further cause the apparatus to:
determine an ambient lighting intensity; and
adjust, based on the ambient lighting intensity, the CFF.

18. The apparatus of claim 12, wherein the processor executable instructions, when executed by the one or more processors, further cause the apparatus to:
store, in a user profile, at least one of: the CFF as a portion of a historical record of CFF's, a light emission frequency variation, an average critical flicker frequency (CFF), or an examination schedule.

19. The apparatus of claim 12, wherein the processor executable instructions, when executed by the one or more processors, further cause the apparatus to determine that a light source is aligned with a user's vision, prior to causing light to be emitted.

20. The apparatus of claim 12, wherein the processor executable instructions, when executed by the one or more processors, further cause the apparatus to discard the CFF based on a cutoff value.
